(19) United States
(12) Patent Application Publication
(10) Pub. No.: US 2021/0212527 A1
(43) Pub. Date: Jul. 15, 2021

(54) DEVICE FOR ASSISTING IN SITTING ON AND GETTING UP FROM TOILET STOOL AND HAVING COMMUNICATION MODULE FOR TRANSMITTING EMERGENCY CALL SIGNAL

(71) Applicant: LINK OPTICS CORP., Gwangju (KR)

(72) Inventor: Yong Won CHOI, Gwangju (KR)

(21) Appl. No.: 17/055,225

(22) PCT Filed: Jun. 26, 2019
(86) PCT No.: PCT/KR2019/007693

§ 371 Date: Nov. 13, 2020

(30) Foreign Application Priority Data
Jun. 27, 2018 (KR) 10-2018-0073893
May 28, 2019 (KR) 10-2019-0062528

(51) Int. Cl. A47K 13/10; H01L 33/00; A47K 17/02; G01N 33/493; A61G 7/10; G08B 25/00; G08B 3/10

(52) U.S. Cl. CPC A47K 13/10; H01L 33/0041; A47K 17/026; G01N 27/4145; A61G 7/1007; G08B 25/00; G08B 3/10; G01N 33/493

(57) ABSTRACT

A device according to an embodiment of the present disclosure is for assisting in sitting on and getting up from a toilet stool and having a communication module for transmitting an emergency call signal. The device assists in sitting on and getting up from the toilet stool which is provided in a restroom for users with restricted mobility, such as the elderly and the weak, patients with arthritis, patients with spinal disorders, etc. More particularly, the device assists in sitting on and getting up from the toilet stool which assists the restroom users to sit on or get up from the toilet stool so that the users may use the toilet stool more conveniently and safely, and has a communication module provided therewith for the users to transmit an emergency call signal that may notify a guardian or a relevant organization when an emergency occurs.

### TECHNICAL FIELD

The present disclosure relates to a device for assisting in sitting on and getting up from a toilet stool provided in a restroom for users with reduced mobility, such as the elderly and the weak, arthritis patients, and spinal disorder patients. More particularly, the present disclosure relates to a device for assisting in sitting on and getting up from a toilet stool and having a communication module for transmitting an emergency call signal, wherein the device assists a restroom user to sit on or get up from the toilet stool so as to use the toilet stool more conveniently and safely, and enables the user himself or herself to notify a guardian, or a relevant organization when an emergency situation occurs.

### BACKGROUND ART

As for a general toilet stool, a seat plate is mounted horizontally at a certain height on an upper part of a toilet body, and due to a structure of the toilet stool, there is a problem in that it is difficult for the elderly and the weak with reduced strength in the legs, arthritis patients, or patients with reduced mobility to not only sit on the toilet stool, but get up again after sitting on the toilet stool.

Since use of the toilet is inevitably uncomfortable for the elderly and the weak, or patients with reduced mobility, especially those suffering from spinal disorders, and help from caregivers around is in fact needed in a process of sitting on or getting up from the toilet stool as well as moving to the toilet, there is a problem in that the caregivers should always be around to help.

In consideration of such conventional problems, various toilet stool devices have been developed so that people with reduced mobility may use the toilet stool conveniently and safely, but there are problems in that the toilet stool devices for helping the sitting and getting up still have many aspects insufficient to help users with reduced mobility to get up, and a number of products have complex structures of their own, causing risk of various safety accidents in some cases.

### DISCLOSURE

### Technical Problem

The present disclosure aims to solve the conventional problems as described above, and an objective of the present disclosure is to provide a device for assisting in sitting on and getting up from a toilet stool and having a communication module for transmitting an emergency call signal, wherein the device is provided in the toilet stool in a restroom for users with reduced mobility such as the elderly and the weak, arthritis patients, and patients with spinal disorders so as to assist in sitting or getting up of the users using the toilet stool in the restroom, improving ease of use and safety, and the device transmits an emergency call signal when an emergency situation occurs.

In addition, another objective of the present disclosure is to provide a device for assisting in sitting on and getting up from a toilet stool and having a communication module for transmitting an emergency call signal, wherein a biomarker (i.e., indicator material) related to a specific disease may be easily detected by amplifying a sensor signal by using a plasmon nanostructure layer containing gold nanoparticles on the surface of a plasmon resonance biosensor.

### Technical Solution

According to the present disclosure to achieve the above objectives, a device for assisting in sitting on and getting up from a toilet stool and having a communication module for transmitting an emergency call signal includes: a pair of housings respectively installed on left and right sides of the toilet stool; a lifting plate installed to be mounted and detachable on an upper part of each housing and connected to a seat installed on the toilet stool for a user to be able to sit on the toilet stool; an armrest installed on the lifting plate or each housing to be able to support user's arms; a lifting driver installed in each housing and having one side thereof connected to the lifting plate so that when the user sitting on the seat gets up, the seat is lifted inclined toward upper and front sides of the toilet stool, making a rear end side of the seat to be higher than a front end side thereof, and when the user sits on the seat, the seat is lowered inclined toward the toilet stool so that the seat lifted to upper and front sides of the seat is positioned horizontally on the toilet stool; an operation part comprising a plurality of operation keys for supporting to control an operation of the lifting driver by a manipulation of the user; and a controller for controlling the operation of the lifting driver according to an input signal input to the operation part.

The device for assisting in sitting on and getting up from a toilet stool and having a communication module for transmitting an emergency call signal further includes a communication module for transmitting the emergency call signal to a guardian of the user or a relevant organization when an emergency situation occurs to the user, wherein the operation part comprises a call key for generating the emergency call signal by the manipulation of the user.

The lifting driver may include: a driving cylinder installed inside each housing; and a lifting rod having one end thereof connected to the driving cylinder and having an opposite end thereof connected to the lifting plate so that the lifting plate is lifted or lowered while being drawn out to the upper part of each housing or drawn into an inner part of each housing by an operation of the driving cylinder, wherein the lifting rod may be formed in a shape of a curve having a predetermined curvature.

The device for assisting in sitting on and getting up from a toilet stool and having a communication module for transmitting an emergency call signal may further include: a sensor part comprising a load detection sensor detecting a load of the driving cylinder, or comprising a pressure detection sensor detecting that an object or a part of a user's body is caught between the toilet stool and the seat; and an alarm part transmitting a warning sound when a load higher than a reference load set by the load detection sensor is detected, or when a pressure higher than a reference pressure is detected by the pressure detection sensor.

The device for assisting in sitting on and getting up from a toilet stool and having a communication module for transmitting an emergency call signal may further include: a plurality of biosensor modules that outputs a reference signal including a voltage signal or a current signal by light incident from the outside, but outputs a reaction signal different from the reference signal when reacting with a biomarker contained in urine or body fluid; a detection module for receiving the reaction signal output from each of the biosensor modules and specifying a type of the biomarker reacted with each of the biosensor modules through the received reaction signal; and a plurality of light emitting modules for emitting the light of different colors according to the type of the biomarker specified in the detection module, wherein each of the biosensor modules may include: a substrate part being transparent and flexible; a gate electrode formed on the substrate part; an oxide layer formed on the gate electrode; an N-type semiconductor layer formed on the oxide layer; a source electrode formed on one side of the oxide layer to contact the oxide layer and the N-type semiconductor layer; a drain electrode formed on an opposite side of the oxide layer facing the source electrode to contact the oxide layer and the N-type semiconductor layer; and a plasmon nanostructure layer disposed on the n-type semiconductor layer and comprising gold nanoparticles that emit free electrons excited by the light incident from the outside to the n-type semiconductor layer.

### Advantageous Effects

The device for assisting in sitting on and getting up from a toilet stool and having a communication module for transmitting an emergency call signal according to the present disclosure has an advantage in that the device is provided in the toilet stool in a restroom for users with reduced mobility such as the elderly and the weak, arthritis patients, and patients with spinal disorders so as to assist in sitting or getting up of the users using the toilet stool in the restroom, improving such as ease of use and safety, and the device transmits an emergency call signal when an emergency situation occurs, enabling the users to quickly receive a help from a guardian, a relevant organization, or a caregiver.

In addition, the present disclosure has an advantage in that the biomarker (i.e., indicator material) related to the specific disease may be easily detected by amplifying the sensor signal by using the plasmon nanostructure layer containing gold nanoparticles on the surface of the plasmon resonance biosensor.

### DESCRIPTION OF DRAWINGS

FIG. 1 is a perspective view of a device for assisting in sitting on and getting up from a toilet stool and having a communication module for transmitting an emergency call signal according to a first exemplary embodiment of the present disclosure.

FIG. 2 is a side view of the device for assisting in sitting on and getting up from the toilet stool having the communication module for transmitting the emergency call signal according to the first exemplary embodiment of the present disclosure.

FIG. 3 is a block diagram showing a control system of the device for assisting in sitting on and getting up from the toilet stool having the communication module for transmitting the emergency call signal according to the first exemplary embodiment of the present disclosure.

FIG. 4 is a perspective view of a device for assisting in sitting on and getting up from a toilet stool and having a communication module for transmitting an emergency call signal according to a second exemplary embodiment of the present disclosure.

FIG. 5 is a cross-sectional view showing an ozone removing unit of the device for assisting in sitting on and getting up from the toilet stool having the communication module for transmitting the emergency call signal according to the second exemplary embodiment of the present disclosure.

FIG. 6 is a block diagram showing a control system of the device for assisting in sitting on and getting up from the toilet stool having the communication module for transmitting the emergency call signal according to the second exemplary embodiment of the present disclosure.

FIG. 7 is a perspective view of a device for assisting in sitting on and getting up from a toilet stool and having a communication module for transmitting an emergency call signal according to a third exemplary embodiment of the present disclosure.

FIG. 8 is a cross-sectional view showing a biosensor of the device for assisting in sitting on and getting up from the toilet stool having the communication module for transmitting the emergency call signal according to the third exemplary embodiment of the present disclosure.

FIG. 9 is a block diagram showing a control system of the device for assisting in sitting on and getting up from the toilet stool having the communication module for transmitting the emergency call signal according to the third exemplary embodiment of the present disclosure.

### BEST MODE

Hereinafter, with reference to the accompanying drawings, a detailed description will be given of a device for assisting in sitting on and getting up from a toilet stool and having a communication module for transmitting an emergency call signal according to a preferred exemplary embodiment of the present disclosure.

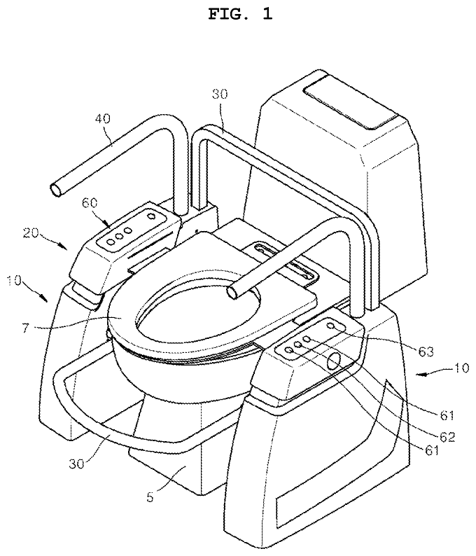

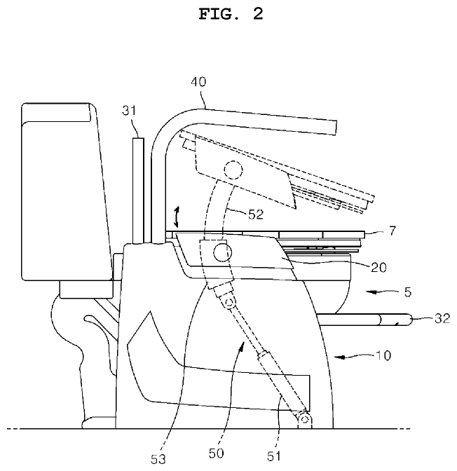

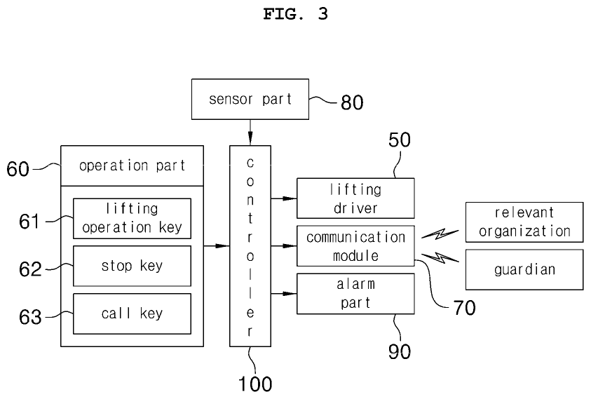

FIGS. 1 to 3 show the exemplary embodiment of the device for assisting in sitting on and getting up from the toilet stool having the communication module for transmitting the emergency call signal according to the present disclosure. Referring to FIGS. 1 to 3, the device for assisting in sitting on and getting up from the toilet stool having the communication module for transmitting the emergency call signal according to the present disclosure is provided with a housing 10, a lifting plate 20, a connecting rod 30, an armrest 40, a lifting driver 50, an operation part 60, a communication module 70, a sensor part 80, an alarm part 90, and a controller 100.

The housing 10 is respectively installed on the left and right sides of the toilet stool 5, and is configured as a pair of housings 10.

Each housing 10 has a space formed therein so as to accommodate the lifting driver 50 and the controller 100, which will be described later. In addition, although not shown in the drawings, a wheel member may be further provided at a lower part of each housing 10 so that each housing 10 may be moved by dragging.

The lifting plate 20 is mounted and detachably installed on an upper part of each housing 10 and is lifted by the lifting driver 50 to be described later.

In addition, the lifting plate 20 of the housing 10 at one side and the lifting plate 20 of the housing 10 at an opposite side are respectively connected to opposite sides of the seat 7 installed on the toilet stool 5 for a user to sit on the toilet stool 5, thereby being lifted together with the seat 7.

The connecting rod 30 is to fix a gap between the housings 10, and one end of the connecting rod 30 is connected to a rear side of the housing 10 at one side and an opposite end of the connecting rod 30 extends to a rear end of the housing 10 the opposite side. The connecting rod 30 connects the housings 10 to each other so that the gap between the housings 10 is greater than the left and right width of the toilet stool 5.

As shown, the connecting rod 30 includes: a first connecting rod 31 having opposite ends thereof fixed to the upper part of each housing 10 corresponding to the rear side of the toilet stool 5 and extending to be bent toward an upper part of the toilet stool 5; and a second connecting rod 32 having opposite ends thereof fixed to side surfaces of each housing 10 facing the toilet stool 5 and extending to be bent toward a front side of each housing 10.

Each armrest 40 is fixedly installed on each lifting plate 20 or each housing 10 so as to support the user's arms to be placed thereon. In the present exemplary embodiment, each armrest 40 that is installed on each housing 10 is applied, but the armrest 40 may be installed on the lifting plate 20 so as to be lifted together with the lifting plate 20.

The lifting driver 50 is installed in each housing 10 and has one side thereof connected to the lifting plate 20 so that when a user sitting on the seat 7 gets up, the seat 7 is lifted inclined toward upper and front sides of the toilet stool 5, making the rear side of the seat 7 to be higher than the front end side, and when the user sits on the seat 7, the seat 7 is lowered inclined toward the toilet stool 5 side so that the seat 7 lifted to the upper and front sides of the seat 7 is positioned horizontally on the toilet stool 5 or mounted on the toilet stool 5.

The lifting driver 50 is configured to include a driving cylinder 51 and a lifting rod 52.

One end of the driving cylinder 51 is fixedly installed inside each housing 10, and an opposite end of the driving cylinder 51 is rotatably connected to the lifting rod 52, so that one side of the lifting rod 52 is drawn out to an outer part of the housing 10, or is drawn into an inner part of the housing 10.

One end of the lifting rod 52 is rotatably connected to the drive cylinder 51, and an opposite end of the lifting rod 52 is connected to the lifting plate 20, so that the lifting plate 20 is lifted or lowered while being drawn out to the upper part of the housing 10 or drawn into the inner part of the housing 10 by the operation of the driving cylinder 51. The lifting rod 52 is drawn out to the upper part of the housing 10 or is drawn into the inner part of housing 10 through an entry hole provided at the upper part of the housing 10.

As mentioned above, the lifting rod 52 is formed in a shape of a curve having a predetermined curvature so that when the user sitting on the seat 7 gets up, the seat 7 is lifted inclined to upper and front sides of the toilet stool 5, making a rear end side of the seat 7 to be higher than the front end side, and when the user sits on the seat 7, the seat 7 is lowered inclined toward the toilet stool 5 so that the seat 7 lifted to the upper and front sides of the seat 7 is positioned horizontally on the toilet stool 5 or mounted on the toilet stool 5.

Inside the housing 10, a guide 53 for guiding the lifting rod 52 may be further provided therein so that the lifting plate 20 and the seat 7 may obliquely protrude or be drawn into the front side of the toilet stool 5 when the lifting rod 52 is drawn out to the outer part of the housing 10 or is drawn into to the inner part of the housing 10.

One end of the guide 53 is fixed to an entrance side provided in the housing 10 through which the lifting rod 52 enters and exits, has a guide hole formed therein so as to allow the fixed lifting rod 52 to pass therethrough, is formed in a shape corresponding to the shape of the lifting rod 52, and is formed to be shorter than the length of the lifting rod 52.

The operation part 60 is configured to control the operation of the lifting driver 50 by a user's operation, and may be installed on the housing 10 or the lifting plate 20. The operation part 60 installed on the lifting plate 20 is applied in the present exemplary embodiment.

The operation part 60 is configured to include a plurality of lifting operation keys 61 for controlling the operation of the lifting driver 50. The lifting operation key 61 is configured to include: a lifting operation key for lifting the seat 7; a lowering operation key for lowering the seat 7; and a stop key 62 for emergency stop during lifting or lowering.

The controller 100 controls the operation of the lifting driver 50 according to the user's input signal input to the operation part 60.

The communication module 70 transmits an emergency call signal to a guardian of a user or a relevant organization when an emergency situation occurs to the user. To this end, the controller 100 stores a phone number of the guardian or the relevant organization in advance.

In addition, the operation part 60 is further provided with a call key 63 for generating the emergency call signal by a user's operation.

The sensor part 80 includes a load detection sensor that detects load of the driving cylinder 51 or a pressure detection sensor that detects that an object or a part of the user's body is caught between the toilet stool 5 and the seat 7. Load information and pressure information sensed by the sensor part 80 are transmitted to the controller 100.

The alarm part 90 transmits a warning sound to the user on the spot by the controller 100 when a load higher than a reference load set by the load detection sensor is detected or a pressure higher than a reference pressure is detected by the pressure detection sensor. In addition, the controller 100 controls the lifting driver 50 so that the lifting driver 50 is immediately stopped when the load higher than the reference load set by the load detection sensor is detected or the pressure higher than the reference pressure is detected by the pressure detection sensor.

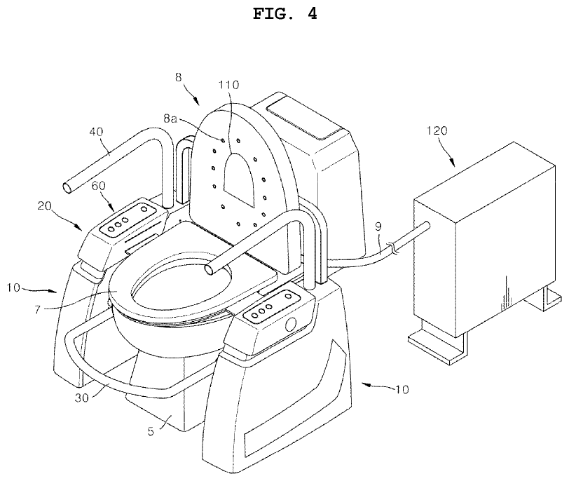

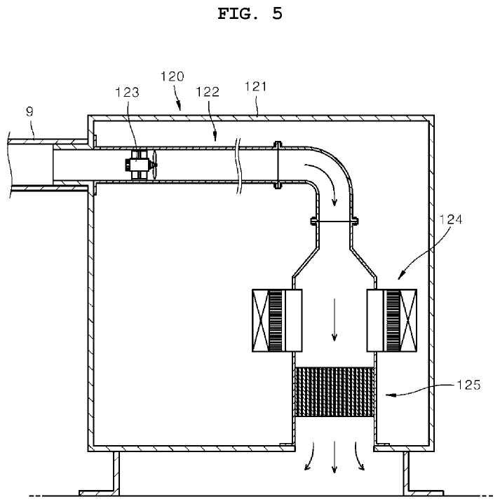

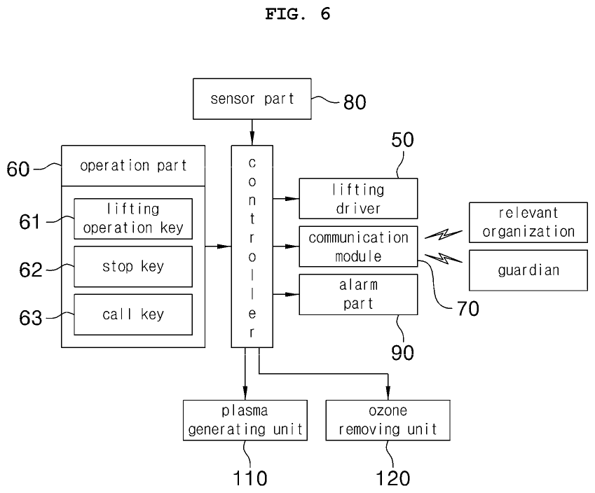

Meanwhile, in FIGS. 4 to 6, it is shown that the device for assisting in sitting on and getting up from a toilet stool and having a communication module for transmitting an emergency call signal according to another exemplary embodiment of the present disclosure is further provided with a plasma generating unit 110 for sterilizing the toilet stool and an ozone removing unit 120 for removing ozone.

Referring to FIGS. 4 to 6, the plasma generating unit 110 is installed on one side of the cover 8 rotatably installed on the rear end side of the seat 7 to open and close an opening of the seat 7, and generates plasma for sterilizing a space inside the toilet stool 5.

The plasma generating unit 110 is configured to include: a first electrode; a second electrode disposed to face the first electrode; and an insulator interposed between the first electrode and the second electrode and having a net or porous structure.

The ozone removing unit 120 is for suctioning and removing ozone generated in the space inside the toilet stool 5 by the plasma generated in the plasma generating unit 110, and is configured to include a casing 121, a duct part 122, a suction fan 123, a heater 124, and an ozone remover 125.

The casing 121 is formed in a screen structure in which a lower part thereof is open and a plurality of holes is formed on the side surfaces thereof.

In order to install the ozone removing unit 120, a suction space is formed inside the cover 8, and a plurality of suction holes on one side of the cover 8 on which the plasma generating unit 110 is installed is formed.

The duct part 122 is installed to extend within the casing 121, is connected to an opposite end of a connecting pipe having an end thereof connected to a rear end of the cover 8 so as to communicate with the suction space of the cover 8, is provided with a flow path formed along the inside of the duct part, and is installed so that an end thereof communicates with the opening formed in the lower part of the casing 121.

The suction fan 123 is installed in the duct part 122 so that ozone generated together with the plasma in the space inside the toilet stool 5 may be suctioned into the duct part 122 through the connecting pipe and be discharged to the outside.

The heater 124 is installed inside the duct part 122 to heat air flowing along the flow path of the duct part 122, so as to deoxidize ozone, and a Peltier element is applied in the present exemplary embodiment.

In the Peltier element, a heating side that generates heat when applying an electric current is disposed to face the inside of the duct part 122, a cooling side that is cooled when applying the electric current is disposed to be exposed to the outside of the duct part 122, and the cooling side is further provided with a heat sink and a heat radiation fan.

The Peltier element is applied to the heater 124, but unlike this, a PTC heater may be applied to the heater, or a heater using a heating wire may be applied as well.

The ozone remover 125 is configured to be installed inside the duct part 122 to allow the air flowing along the flow path of the duct part 122 to pass therethrough, and is supplied with a manganese dioxide catalyst decomposing and removing ozone contained in the air by deoxidizing the ozone.

As shown, the ozone remover 125 causes manganese dioxide to be formed in a cylindrical block, and may be formed in a structure in which a plurality of through holes are formed along a length direction. In addition, unlike shown, the ozone remover 125 may be formed in a shape in which manganese dioxide is formed in a pellet shape, and the pellet is contained in a fine mesh net.

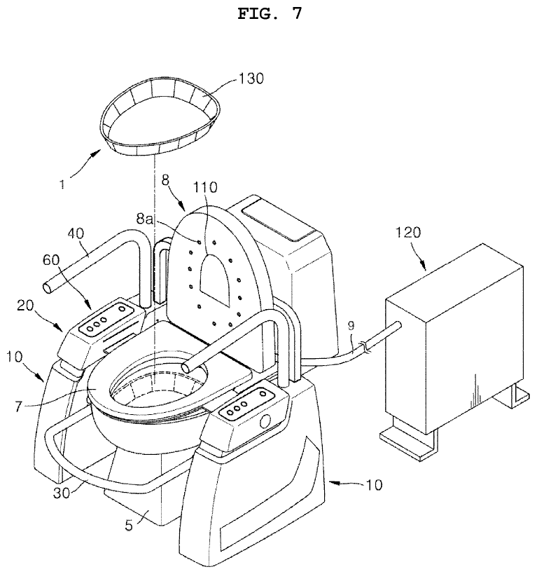

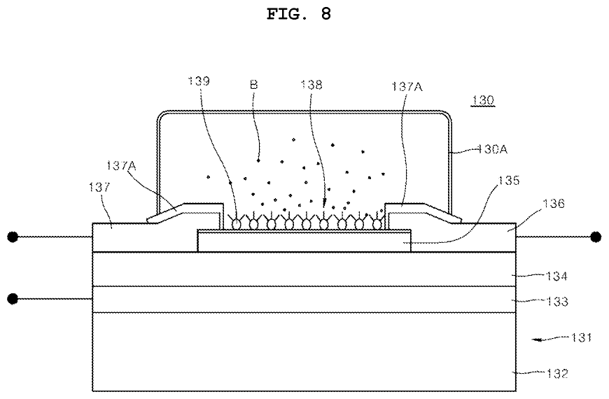

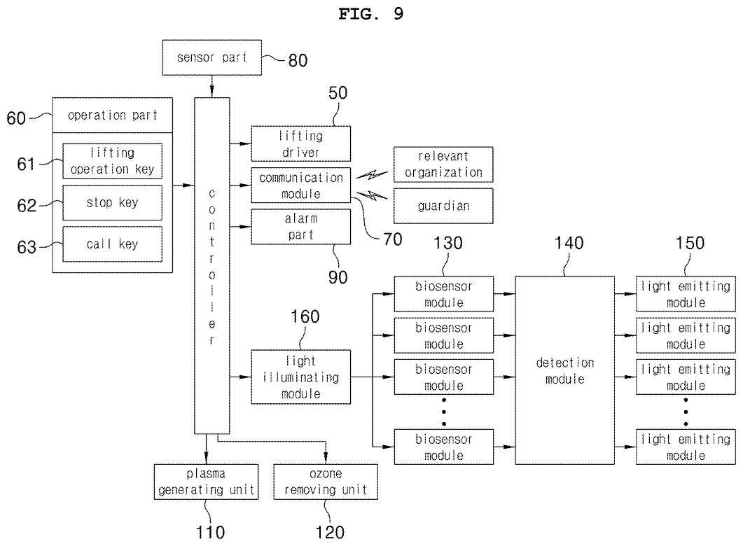

Meanwhile, in FIGS. 7 to 9, it is shown that a device for assisting in sitting on and getting up from a toilet stool and having a communication module for transmitting an emergency call signal according to yet another exemplary embodiment of the present disclosure further includes: a plurality of biosensor modules 130 that outputs a reference signal including a voltage signal or a current signal by light incident from the outside, but outputs a reaction signal different from the reference signal when reacting with a biomarker B contained in urine or body fluid; a detection module 140 for receiving a reaction signal output from the biosensor modules 130 and specifying a type of biomarker B reacted with the biosensor modules 130 through the received reaction signal; a plurality of light emitting modules 150 for emitting light of different colors according to the type of biomarker B specified in the detection module 140; and a light illuminating module 160 for illuminating the biosensor modules 130 with excitation light.

Referring to FIGS. 7 to 9, the biosensor modules 130 output the reference signal including the voltage signal or the current signal by the light incident from the outside, but output the reaction signal different from the reference signal when reacting with the biomarker B contained in urine or body fluid.

In the present disclosure, the biomarker B is an indicator material useful for diagnosis of diseases, and as an example, may include: anti-GAD antibody, which is an indicator material for diabetes; PSA protein, which is an indicator material for prostate cancer; Phosphorus AFP, which is an indicator material for liver cancer; and troponin C (TnC), troponin T (TnT), and troponin I (TnI), which are indicator materials related to heart diseases.

In addition, the biomarker may include: myocardial fibrosis, whose concentration is increased by myocardial cell death, fibrosis of myocardial tissue, decreased heart function, and increased heart disease progression; T2 (sST2), which is an indicator of cardiac remodeling; ST2 predicting a possibility of developing hypertension or heart failure in the future in people without heart disease; and the like.

The biosensor modules 130 are installed inside the toilet stool as shown, so that a binding reaction with the biomarker B contained in urine is performed.

The biosensor module 130 is configured to include a plurality of biosensor modules, and opposite ends thereof is respectively connected to adjacent biosensor modules 130 to form a strip shape.

Each of the biosensor modules 130 is provided with an electrode line for transmitting the reaction signal to the detection module 140 to be described later, and the electrode line of each biosensor module 130 is connected to the detection module 140 so as to transmit each independent reaction signal to the detection module 140.

Each biosensor module 130 is configured to include a field effect transistor 131 and a plasmon nanostructure layer 138 formed on an upper part of the field effect transistor 131.

The field effect transistor 131 includes: a substrate part 132 being transparent and flexible so as to allow light to pass therethrough; a gate electrode 133 formed on the substrate part 132; an oxide layer 134 formed on the gate electrode 133; an N-type semiconductor layer 135 formed on the oxide layer 134; a source electrode 136 formed on one side of the oxide layer 134 to contact the oxide layer 134 and the N-type semiconductor layer 135; and a drain electrode 137 formed on the opposite side of the oxide layer 134 facing the source electrode 136 to contact the oxide layer 134 and the N-type semiconductor layer 135.

The gate electrode 133 may be applied with indium tin oxide (ITO), the oxide layer 134 may be applied with silicon dioxide (SiO2), and the n-type semiconductor layer 135 may be applied with zinc oxide (ZnO).

The field effect transistor 131 according to the present exemplary embodiment may be applied with a zinc oxide field effect transistor (ZnO FET).

In addition, the field effect transistor 131 of the biosensor module 130 is not limited to the specific material or compound described above, and may include one or more other materials or compounds that enable the FET to detect electromagnetic energy through plasmon absorption.

In addition, the plasmon nanostructure layer 138 is formed on the n-type semiconductor layer 135 and is configured to include a plurality of gold nanoparticles that emits free electrons excited by light incident from the outside to the n-type semiconductor layer 135.

The plasmon nanostructure layer 138 is formed to maintain a state insulated from each of the drain electrode 137 and the source electrode 136 by a passivation layer 137A.

In the drawing, reference numeral 130A denotes a sample channel through which urine may flow in and out.

In each biosensor module 130 as described above, when light emitted from the outside is incident on the gold nanoparticles 139 of the plasmon nanostructure layer 138 in a state in which binding and reaction with the biomarker B contained in urine is not performed, a plasmon resonance phenomenon in which free electrons in the gold nanoparticles 139 vibrate at the same time occurs, and the free electrons in the gold nanoparticles 139 move into the n-type semiconductor layer 135 in contact with the gold nanoparticles 139.

At this time, the reference signal is generated between the drain electrode 137 and the source electrode 136, and the generated reference signal is transmitted to the detection module 140 to be described later. In addition, the reference signal received by the detection module 140 is stored in advance in the detection module 140 and used for comparison with the reaction signal to be described later.

Meanwhile, when urine adheres to the plasmon nanostructure layer 138 of the biosensor module 130 so that the biomarker B contained in the urine reacts with the gold nanoparticles 139 of the plasmon nanostructure layer 138, a reaction signal different from the reference signal described above is generated between the drain electrode 137 and the source electrode 136, whereby the generated reaction signal is transmitted to the detection module 140 to be described later.

The detection module 140 receives the reaction signal output from the biosensor, compares the previously stored reference signal with the received reaction signal to specify the type of biomarker B reacted with each biosensor module 130, and controls the operation of the light emitting module 150 to be described later according to the type of the specified biomarker B. To this end, the detection module 140 may be provided with a current detector or a voltage detector.

The light emitting module 150 is configured to include a plurality of light emitting modules to emit light of different colors according to the type of biomarker B specified by the detection module 140, and is provided with a plurality of micro LEDs emitting different light according to the number of biomarkers B to be detected.

The plurality of micro LEDs correspond one-to-one with the specific biomarkers B, and selectively operate according to the type of biomarker B detected by the detection module 140.

The light illuminating module 160 is installed inside the toilet stool in which the biosensor modules 130 are installed or installed at an inner edge of the seat to provide excitation light emitting toward the biosensor.

The device for assisting in sitting on and getting up from the toilet stool having the communication module for transmitting the emergency call signal according to the present disclosure described above has been described with reference to the accompanying drawings, but this is only exemplary, and those skilled in the art will be understood that various modifications and equivalent other exemplary embodiments may be realized therefrom.

Therefore, the scope of the true technical protection of the present disclosure should be determined only by the technical spirit of the appended claims.

1. A device for assisting in sitting on and getting up from a toilet stool, the device comprising:
a pair of housings respectively installed on left and right sides of the toilet stool;
a lifting plate installed to be mounted and detachable on an upper part of each housing and connected to a seat installed on the toilet stool for a user to be able to sit on the toilet stool;
an armrest installed on the lifting plate or each housing to be able to support user's arms;
a lifting driver installed in each housing and having one side thereof connected to the lifting plate so that when the user sitting on the seat gets up, the seat is lifted inclined toward upper and front sides of the toilet stool, making a rear end side of the seat to be higher than a front end side thereof, and when the user sits on the seat, the seat is lowered inclined toward the toilet stool so that the seat lifted to upper and front sides of the seat is positioned horizontally on the toilet stool;
an operation part comprising a plurality of operation keys for supporting to control an operation of the lifting driver by a manipulation of the user;
a controller for controlling the operation of the lifting driver according to an input signal input to the operation part; and
a communication module for transmitting an emergency call signal to a guardian of the user or a relevant organization when an emergency situation occurs to the user,
wherein the operation part comprises a call key for generating the emergency call signal by the manipulation of the user.

2. The device of claim 1, wherein the lifting driver comprises:
a driving cylinder installed inside each housing; and
a lifting rod having one end thereof connected to the driving cylinder and having an opposite end thereof connected to the lifting plate so that the lifting plate is lifted or lowered while being drawn out to the upper part of each housing or drawn into an inner part of each housing by an operation of the driving cylinder,
wherein the lifting rod is formed in a shape of a curve having a predetermined curvature.

3. The device of claim 2, further comprising:
a sensor part comprising a load detection sensor detecting a load of the driving cylinder, or comprising a pressure detection sensor detecting that an object or a part of a user's body is caught between the toilet stool and the seat; and
an alarm part transmitting a warning sound when a load higher than a reference load set by the load detection sensor is detected, or when a pressure higher than a reference pressure is detected by the pressure detection sensor.

4. The device of claim 1, further comprising:
a cover rotatably installed on the rear end side of the seat to open and close an opening of the seat; and
a plasma generating module installed on one surface of the cover to generate plasma for sterilizing a space inside the toilet stool,
wherein the plasma generating module is installed on one surface of the cover to generate the plasma in the space inside the toilet stool, and the plasma generating module comprises:
a first electrode;
a second electrode disposed to face the first electrode; and
an insulator interposed between the first electrode and the second electrode and having a net or porous structure.

5. The device of claim 4,
wherein a suction space is formed inside the cover, a plurality of suction holes is formed on one surface of the cover on which the plasma generating unit is installed; and
the device further comprises an ozone removing unit, the ozone removing unit comprising:
a duct part having one end thereof connected to a rear end side of the cover to communicate with the suction space of the cover and having a flow path formed along inside the duct part;
a suction fan installed in the duct part so that the ozone generated together with the plasma in the space inside the toilet stool is suctioned into the duct part to be discharged to outside;
a heater installed inside the duct part to heat air flowing along the flow path of the duct part, deoxidizing the ozone; and
an ozone remover installed inside the duct part and formed to allow the air flowing along the flow path of the duct part to pass therethrough and being provided with manganese dioxide catalyst for decomposing and removing the ozone contained in the air by deoxidizing the ozone.

6. The device of claim 1, further comprising:
a plurality of biosensor modules that outputs a reference signal including a voltage signal or a current signal by light incident from the outside, but outputs a reaction signal different from the reference signal when reacting with a biomarker contained in urine or body fluid;
a detection module for receiving the reaction signal output from each of the biosensor modules and specifying a type of the biomarker reacted with each of the biosensor modules through the received reaction signal; and
a plurality of light emitting modules for emitting the light of different colors according to the type of the biomarker specified in the detection module,
wherein each of the biosensor modules comprises:
a substrate part being transparent and flexible;
a gate electrode formed on the substrate part;
an oxide layer formed on the gate electrode;
an N-type semiconductor layer formed on the oxide layer;
a source electrode formed on one side of the oxide layer to contact the oxide layer and the N-type semiconductor layer;
a drain electrode formed on an opposite side of the oxide layer facing the source electrode to contact the oxide layer and the N-type semiconductor layer; and
a plasmon nanostructure layer disposed on the n-type semiconductor layer and comprising gold nanoparticles that emit free electrons excited by the light incident from the outside to the n-type semiconductor layer.
